(19) United States
(12) Patent Application Publication
(10) Pub. No.: US 2013/0211151 A1
(43) Pub. Date: Aug. 15, 2013

(54) Process for Producing Ethanol Using Acetic Acid from a Carbonylation Process

(71) Applicants: Victor J. Johnston, Houston, TX (US); Mark Scates, Houston, TX (US); James Zink, League City, TX (US)

(72) Inventors: Victor J. Johnston, Houston, TX (US); Mark Scates, Houston, TX (US); James Zink, League City, TX (US)

(73) Assignee: CELANESE INTERNATIONAL CORPORATION, Dallas, TX (US)

(21) Appl. No.: 13/371,091

(22) Filed: Feb. 10, 2012

(51) Int. Cl. C07C 29/80

(52) U.S. Cl. 568/885

(57) ABSTRACT

The present invention relates to a process for the production of ethanol. The process comprises the step of hydrogenating acetic acid in a hydrogenation reactor in the presence of a catalyst and under conditions effective to form a crude ethanol product. The acetic acid may be obtained from a carbonylation system. The process further comprises the step of separating, in at least one column, at least a portion of the crude ethanol product into a distillate and a residue. The distillate comprises ethanol, water, and ethyl acetate. The residue comprises acetic acid and water. The process preferably comprises the step of directing at least a portion of the residue to at least one column of the carbonylation system. The process further comprises the step of separating the first distillate to form a purified ethanol product.

### FIELD OF THE INVENTION

The present invention relates generally to processes for recovering ethanol produced by the hydrogenation of acetic acid, ethyl acetate, and mixtures thereof. In particular, the present invention relates to a separation scheme in which a derivative of a crude ethanol product is directed to a drying column of a methanol carbonylation process.

### BACKGROUND OF THE INVENTION

Ethanol for industrial use is conventionally produced from organic feed stocks, such as petroleum oil, natural gas, or coal, from feed stock intermediates, such as syngas, or from starchy materials or cellulose materials, such as corn or sugar cane. Conventional methods for producing ethanol from organic feed stocks, as well as from cellulose materials, include the acid-catalyzed hydration of ethylene, methanol homologation, direct alcohol synthesis, and Fischer-Tropsch synthesis. Instability in organic feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The hydrogenation of alkanoic acid, e.g., acetic acid, yields a crude ethanol product that comprises impurities, e.g., water, which are often formed with ethanol or in side reactions. These impurities may limit the production of ethanol and may require expensive and complex purification trains to separate the impurities from the ethanol.

Some processes for integrating acetic acid production and hydrogenation have been proposed in literature.

For example, U.S. Pat. No. 7,884,253 discloses methods and apparatuses for selectively producing ethanol from syngas. The syngas is derived from cellulosic biomass (or other sources) and can be catalytically converted into methanol, which in turn can be catalytically converted into acetic acid or acetates. The ethanoic acid product may be removed from the reactor by withdrawing liquid reaction composition and separating the ethanoic acid product by one or more flash and/or fractional distillation stages from the other components of the liquid reaction composition such as iridium catalyst, ruthenium and/or osmium and/or indium promoter, methyl iodide, water and unconsumed reactants which may be recycled to the reactor to maintain their concentrations in the liquid reaction composition. As another example, EP2060553 discloses a process for the conversion of a carbonaceous feedstock to ethanol wherein the carbonaceous feedstock is first converted to ethanoic acid, which is then hydrogenated and converted into ethanol. Also, U.S. Pat. No. 4,497,967 discloses an integrated process for the preparation of ethanol from methanol, carbon monoxide and hydrogen feedstock. The process esterifies an acetic anhydride intermediate to form ethyl acetate and/or ethanol. In addition, U.S. Pat. No. 7,351,559 discloses a process for producing ethanol including a combination of biochemical and synthetic conversions resulting in high yield ethanol production with concurrent production of high value co-products. An acetic acid intermediate is produced from carbohydrates, such as corn, using enzymatic milling and fermentation steps, followed by conversion of the acetic acid into ethanol using esterification and hydrogenation reactions.

One conventional process for preparing acetic acid is methanol carbonylation, which reacts methanol and carbon monoxide to form acetic acid. Typically, methanol carbonylation processes form a crude acetic acid product, which is then purified in a separation zone. The separation zone may comprise one or more columns, e.g., a light ends column and/or a drying column.

In view of the conventional processes and literature, the need remains for improved ethanol production processes that are capable of 1) effectively separating the crude ethanol product to remove impurities, including water; and 2) capturing unreacted acetic acid.

### SUMMARY OF THE INVENTION

The present invention relates to a process for the production of ethanol. The process comprises the step of hydrogenating acetic acid in a hydrogenation reactor in the presence of a catalyst and under conditions effective to form a crude ethanol product. The acetic acid may be obtained from a carbonylation system. The process further comprises the step of separating, in at least one column, at least a portion of the crude ethanol product into a distillate and a residue. The distillate comprises ethanol, water, and ethyl acetate. The residue comprises acetic acid and water. The process preferably comprises the step of directing at least a portion of the residue to at least one column of the carbonylation system. The process further comprises the step of separating the distillate to form a purified ethanol product.

### BRIEF DESCRIPTION OF DRAWINGS

The invention is described in detail below with reference to the appended drawings, wherein like numerals designate similar parts.

FIG. 1 is a flowsheet of a carbonylation and hydrogenation process in accordance with an embodiment of the present invention.

FIG. 2 is a schematic diagram of a carbonylation and hydrogenation process in accordance with an embodiment of the present invention.

FIG. 3 is a schematic diagram of a carbonylation and hydrogenation process in accordance with an embodiment of the present invention.

FIG. 4 is a schematic diagram of a carbonylation and hydrogenation process in accordance with an embodiment of the present invention.

FIG. 5 is a schematic diagram of a carbonylation and hydrogenation process in accordance with an embodiment of the present invention.

### DETAILED DESCRIPTION OF THE INVENTION

### Introduction

Acetic acid may be formed via the carbonylation of methanol. In this reaction, carbon monoxide and methanol are reacted to form the acetic acid. Typically, methanol carbonylation processes form a crude acetic acid product, which is then purified in a methanol carbonylation separation zone. The carbonylation separation zone may comprise one or more columns, e.g., a light ends column and/or a drying column. Conventional carbonylation separation zones separate the crude acetic acid product to form one or more derivative streams comprising acetic acid and water. Typically, these water-containing derivative streams are directed to the drying column wherein the water is separated from the acetic acid. The drying column may be very efficient in removing water to less than 1500 wppm as well as other impurities.

Acetic acid may be hydrogenated to form a crude ethanol product. The crude ethanol product comprises, inter alia, ethanol, acetic acid, and water. The crude ethanol product is typically separated in a hydrogenation separation zone to form one or more derivative streams. In some cases, the derivative streams may comprise acetic acid and water. Such streams conventionally require additional separation units to separate these components from one another.

In one embodiment, a crude ethanol derivative stream comprising acetic acid and water may be directed to a drying column of the carbonylation separation zone. In the drying column, the water, along with any other impurities, may be separated from the acetic acid. In one embodiment the crude ethanol derivative stream may be directed to light ends column and/or the drying column of the carbonylation separation zone. The acetic acid may also be recovered and returned to the hydrogenation reactor to be converted to ethanol. By utilizing the drying column of the carbonylation separation zone to separate a derivative stream, the need for a separate column to separate acetic acid and water may be reduced or eliminated.

Accordingly, the present invention, in one embodiment, relates to a process for producing ethanol. Ethanol may be produced from acetic acid obtained by carbonylating methanol. The process comprises the step of hydrogenating acetic acid in the presence of a catalyst and under conditions effective to form a crude ethanol product. The hydrogenation reaction may be conducted in a hydrogenation reactor. Preferably, the acetic acid is obtained from a carbonylation system, which may comprise a carbonylation reaction zone and a carbonylation separation zone. The carbonylation separation zone preferably comprises at least one column, e.g., a light ends column and/or a drying column. The process further comprises the step of separating at least a portion of the crude ethanol product into a distillate and a residue. The distillate may comprise ethanol, water, and ethyl acetate. The residue may comprise acetic acid and water. In one embodiment, the separation is achieved in a hydrogenation separation zone that comprises at least one column, e.g., an acid separation column. Preferably, the process further comprises the step of directing at least a portion of the residue to at least one column of the carbonylation system. For example, the residue may be directed to the drying column of the carbonylation separation zone. In a preferred embodiment, substantially none of the residue is directly fed to a hydrogenation reactor, e.g., substantially all of the residue is directed to the carbonylation separation zone. In one embodiment, the drying column of the carbonylation separation zone separates the residue to form a purified acetic acid stream and a water stream. Preferably, the purified acetic acid stream comprises less than 1500 wppm water, e.g., less than 1000 wppm or less than 500 wppm. In terms of ranges the purified acetic acid stream may comprise from 1 wppm to 1500 wppm water, e.g., from 1 wppm to 1000 wppm or from 100 wppm to 500 wppm. The process further comprises the step of separating the distillate to form a purified ethanol product.

The composition of the residue may vary depending the ethanol separation process. Any (residue) stream that primarily comprises acetic acid and water may be fed to the drying column of the carbonylation process. In some embodiments, the residue may also comprise other organic impurities such as ethyl acetate, and aldehyde. In one embodiment, the residue comprises from 60 wt. % to 99 wt. % acetic acid, e.g., from 70 wt. % to 95 wt. % or from 85 wt. % to 92 wt. %, and from 1 wt. % to 30 wt. % water, e.g., from 1 wt. % to 20 wt. % or from 1 wt. % to 15 wt. %. In one embodiment, the residue comprises from 1 wt. % to 70 wt. % acetic acid, e.g., from 1 wt. % to 50 wt. % or from 2 wt. % to 35 wt. %, and from 30 wt. % to 99 wt. % water, e.g., from 45 wt. % to 95 wt. % or from 60 wt. % to 90 wt. %. In one embodiment, the residue comprises from 0.1 wt. % to 45 wt. % acetic acid, e.g., from 0.2 wt. % to 40 wt. % or from 0.5 wt. % to 35 wt. % and from 45 wt. % to 99.9 wt. % water, e.g., from 55 wt. % to 99.8 wt. % or from 65 wt. % to 99.5 wt. %.

### Carbonylation

The process of the present invention may be used with any process for producing acetic acid, as long as the separation zone associated therewith comprises at least one column, e.g., a drying column. Preferably, the acetic acid production system is a methanol carbonylation system. Exemplary materials, catalysts, reaction conditions, and separation processes that may be used in the carbonylation of methanol are described further below.

In one embodiment that utilizes carbonylation, the carbonylation system that is employed preferably comprises a reaction zone, which includes a reactor, a flasher and optionally a reactor recovery unit. In one embodiment, carbon monoxide is reacted with methanol in a suitable reactor, e.g., a continuous stirred tank reactor (“CSTR”) or a bubble column reactor. Preferably, the carbonylation process is a low water, catalyzed, e.g., rhodium-catalyzed, carbonylation of methanol to acetic acid, as exemplified in U.S. Pat. No. 5,001,259, which is hereby incorporated by reference.

Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference.

The carbonylation reaction may be conducted in a homogeneous catalytic reaction system comprising a reaction solvent, methanol and/or reactive derivatives thereof, a Group VIII catalyst, at least a finite concentration of water, and optionally an iodide salt. In one embodiment, methanol is obtained from an impure methanol feed that is not purified prior to carbonylation.

Suitable carbonylation catalysts include Group VIII catalysts, e.g., rhodium and/or iridium catalysts. When a rhodium catalyst is utilized, the rhodium catalyst may be added in any suitable form such that the active rhodium catalyst is a carbonyl iodide complex. Exemplary rhodium catalysts are described in Michael Gauβ, et al., Applied Homogeneous Catalysis with Organometallic Compounds: A Comprehensive Handbook in Two Volume, Chapter 2.1, p. 27-200, (1^{st} ed., 1996). Iodide salts optionally maintained in the reaction mixtures of the processes described herein may be in the form of a soluble salt of an alkali metal or alkaline earth metal or a quaternary ammonium or phosphonium salt. In certain embodiments, a catalyst co-promoter comprising lithium iodide, lithium acetate, or mixtures thereof may be employed. The salt co-promoter may be added as a non-iodide salt that will generate an iodide salt. The iodide catalyst stabilizer may be introduced directly into the reaction system. Alternatively, the iodide salt may be generated in-situ since under the operating conditions of the reaction system, a wide range of non-iodide salt precursors will react with methyl iodide or hydroiodic acid in the reaction medium to generate the corresponding co-promoter iodide salt stabilizer. For additional detail regarding rhodium catalysis and iodide salt generation, see U.S. Pat. Nos. 5,001,259; 5,026,908; and 5,144,068, which are hereby incorporated by reference.

When an iridium catalyst is utilized, the iridium catalyst may comprise any iridium-containing compound which is soluble in the liquid reaction composition. The iridium catalyst may be added to the liquid reaction composition for the carbonylation reaction in any suitable form which dissolves in the liquid reaction composition or is convertible to a soluble form. Examples of suitable iridium-containing compounds which may be added to the liquid reaction composition include: IrCl₃, IrI₃, IrBr₃, [Ir(CO)₂I]₂, [Ir(CO)₂Cl]₂, [Ir(CO)₂Br]₂, [Ir(CO)₂I₂]⁻H⁺, [Ir(CO)₂Br₂]⁻H⁺, [Ir(CO)₂I₄]⁻H⁺, [Ir(CH₃)I₃(CO₂]⁻H⁺, Ir₄(CO)₁₂, IrCl₃.3H₂O, IrBr₃.3H₂O, iridium metal, Ir₂O₃, Ir(acac)(CO)₂, Ir(acac)₃, iridium acetate, [Ir₃O(OAc)₆(H₂O)₃][OAc], and hexachloroiridic acid [H₂IrCl₆]. Chloride-free complexes of iridium such as acetates, oxalates and acetoacetates are usually employed as starting materials. The iridium catalyst concentration in the liquid reaction composition may be in the range of 100 to 6000 ppm. The carbonylation of methanol utilizing iridium catalyst is well known and is generally described in U.S. Pat. Nos. 5,942,460; 5,932,764; 5,883,295; 5,877,348; 5,877,347; and 5,696,284, which are hereby incorporated by reference.

A halogen co-catalyst/promoter is generally used in combination with the Group VIII metal catalyst component. Methyl iodide is a preferred halogen promoter. Preferably, the concentration of halogen promoter in the reaction medium ranges from 1 wt. % to 50 wt. %, and preferably from 2 wt. % to 30 wt. %.

The halogen promoter may be combined with the salt stabilizer/co-promoter compound. Particularly preferred are iodide or acetate salts, e.g., lithium iodide or lithium acetate.

Other promoters and co-promoters may be used as part of the catalytic system of the present invention as described in U.S. Pat. No. 5,877,348, which is hereby incorporated by reference. Suitable promoters are selected from ruthenium, osmium, tungsten, rhenium, zinc, cadmium, indium, gallium, mercury, nickel, platinum, vanadium, titanium, copper, aluminum, tin, antimony, and are more preferably selected from ruthenium and osmium. Specific co-promoters are described in U.S. Pat. No. 6,627,770, which is incorporated herein by reference.

A promoter may be present in an effective amount up to the limit of its solubility in the liquid reaction composition and/or any liquid process streams recycled to the carbonylation reactor from the acetic acid recovery stage. When used, the promoter is suitably present in the liquid reaction composition at a molar ratio of promoter to metal catalyst of 0.5:1 to 15:1, preferably 2:1 to 10:1, more preferably 2:1 to 7.5:1. A suitable promoter concentration is 400 to 5000 ppm.

In one embodiment, the temperature of the carbonylation reaction in the reactor is preferably from 150° C. to 250° C., e.g., from 150° C. to 225° C., or from 150° C. to 200° C. The pressure of the carbonylation reaction is preferably from 1 to 20 MPa, preferably 1 to 10 MPa, most preferably 1.5 to 5 MPa. Acetic acid is typically manufactured in a liquid phase reaction at a temperature from about 150° C. to about 200° C. and a total pressure from about 2 to about 5 MPa.

In one embodiment, reaction mixture comprises a reaction solvent or mixture of solvents. The solvent is preferably compatible with the catalyst system and may include pure alcohols, mixtures of an alcohol feedstock, and/or the desired carboxylic acid and/or esters of these two compounds. In one embodiment, the solvent and liquid reaction medium for the (low water) carbonylation process is preferably acetic acid.

Water may be formed in situ in the reaction medium, for example, by the esterification reaction between methanol reactant and acetic acid product. In some embodiments, water is introduced to reactor together with or separately from other components of the reaction medium. Water may be separated from the other components of reaction product withdrawn from reactor and may be recycled in controlled amounts to maintain the required concentration of water in the reaction medium. Preferably, the concentration of water maintained in the reaction medium ranges from 0.1 wt. % to 16 wt. %, e.g., from 1 wt. % to 14 wt. %, or from 1 wt. % to 3 wt. % of the total weight of the reaction product.

The desired reaction rates are obtained even at low water concentrations by maintaining in the reaction medium an ester of the desired carboxylic acid and an alcohol, desirably the alcohol used in the carbonylation, and an additional iodide ion that is over and above the iodide ion that is present as hydrogen iodide. An example of a preferred ester is methyl acetate. The additional iodide ion is desirably an iodide salt, with lithium iodide (LiI) being preferred. It has been found, as described in U.S. Pat. No. 5,001,259, that under low water concentrations, methyl acetate and lithium iodide act as rate promoters only when relatively high concentrations of each of these components are present and that the promotion is higher when both of these components are present simultaneously. The absolute concentration of iodide ion content is not a limitation on the usefulness of the present invention.

In low water carbonylation, the additional iodide over and above the organic iodide promoter may be present in the catalyst solution in amounts ranging from 2 wt. % to 20 wt. %, e.g., from 2 wt. % to 15 wt. %, or from 3 wt. % to 10 wt. %; the methyl acetate may be present in amounts ranging from 0.5 wt % to 30 wt. %, e.g., from 1 wt. % to 25 wt. %, or from 2 wt. % to 20 wt. %; and the lithium iodide may be present in amounts ranging from 5 wt. % to 20 wt %, e.g., from 5 wt. % to 15 wt. %, or from 5 wt. % to 10 wt. %. The catalyst may be present in the catalyst solution in amounts ranging from 200 wppm to 2000 wppm, e.g., from 200 wppm to 1500 wppm, or from 500 wppm to 1500 wppm.

### Hydrogenation of Acetic Acid

The process of the present invention may be used with any hydrogenation process for producing ethanol. The materials, catalysts, reaction conditions, and separation processes that may be used in the hydrogenation of acetic acid are described further below.

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process and/or the methanol carbonylation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

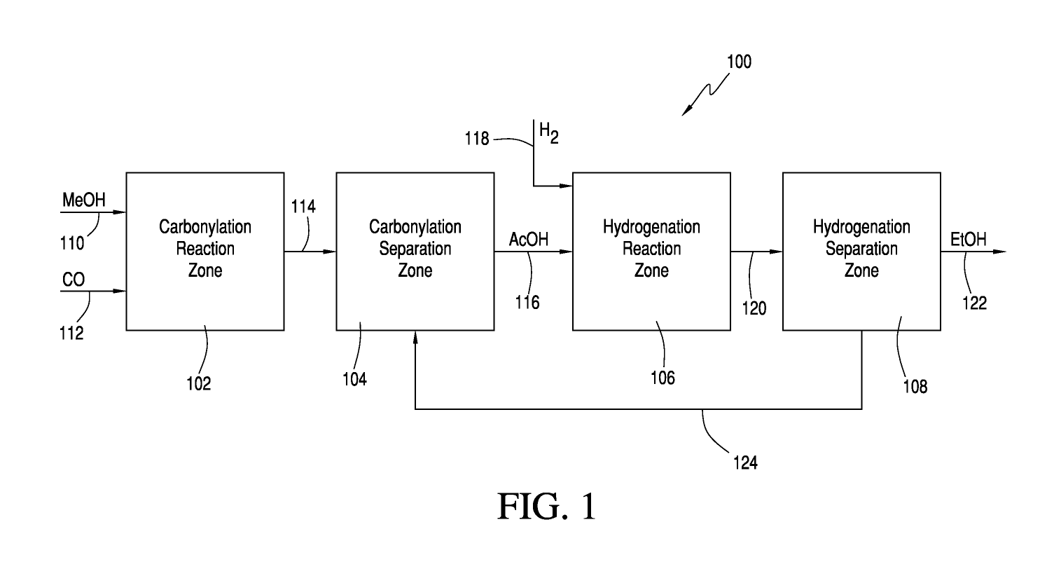

FIG. 1 is a diagram of an integrated process 100 in accordance with the present invention. Process 100 comprises carbonylation reaction zone 102, carbonylation separation zone 104, hydrogenation reaction zone 106, and hydrogenation separation zone 108. Carbonylation reaction zone 102 receives methanol feed 110 and carbon monoxide feed 112. The methanol and the carbon monoxide are reacted in carbonylation reaction zone 102 to form a crude acetic acid product, which comprises acetic acid and impurities and exits carbonylation reaction zone 102 via line 114. Line 114 is directed to carbonylation separation zone 104 wherein the crude acetic acid product is purified. Carbonylation separation zone 104 may comprise a flasher, which may be used to remove residual catalyst from the crude acetic acid product, and at least one column. A purified acetic acid stream exits carbonylation separation zone 104 via line 116. Although not shown, carbonylation separation zone 104 may also yield additional acetic acid-containing streams.

The purified acetic acid product in line 116 is fed, preferably directly fed, to hydrogenation reaction zone 106. Hydrogenation reaction zone 106 also receives hydrogen feed 118. In hydrogenation reaction zone 106, the acetic acid in the purified acetic acid product is hydrogenated to form a crude ethanol product comprising ethanol and other compounds such as water, ethyl acetate, and unreacted acetic acid. The crude ethanol product exits hydrogenation reaction zone 106 via line 120. Hydrogenation separation zone 108 comprises one or more separation units, e.g. distillation columns, (not explicitly shown in FIG. 1) for recovering ethanol from the crude ethanol product. Once separated, a purified ethanol product stream exits hydrogenation separation zone 108 via line 122. Hydrogenation separation zone 106 also yields at least one derivative stream which exits via line 124. The derivative stream(s) may comprise, inter alia, acetic acid and water. Derivative streams(s) 124 are directed to carbonylation separation zone 104 for further processing, as discussed herein.

The hydrogenation reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation in the reactor may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst in the reactor. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIB, VIIB, VIII transition metals, a lanthanide metal, an actinide metal or a metal selected from any of Groups IIIA, IVA, VA, and VIA. Preferred bimetallic combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Additional metal combinations may include palladium/rhenium/tin, palladium/rhenium/cobalt, palladium/rhenium/nickel, platinum/tin/palladium, platinum/tin/cobalt, platinum/tin/copper, platinum/tin/chromium, platinum/tin/zinc, and platinum/tin/nickel.

Exemplary catalysts are further described in U.S. Pat. No. 7,608,744 and U.S. Pub. No. 2010/0029995, the entireties of which are incorporated herein by reference. In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group 111B metal oxides, (viii) Group 111B metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). The calcium metasilicate may be in crystalline or amorphous form.

A preferred silica support material is SS61138 High Surface Area (HSA) Silica Catalyst Carrier from Saint-Gobain N or Pro. The Saint-Gobain N or Pro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from SW Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197985 referred to above, the entireties of which are incorporated herein by reference.

In particular, the hydrogenation of acetic acid may achieve favorable conversion of acetic acid and favorable selectivity and productivity to ethanol in the reactor. For purposes of the present invention, the term “conversion” refers to the amount of acetic acid in the feed that is converted to a compound other than acetic acid. Conversion is expressed as a percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethanol is at least 60%, e.g., at least 70%, or at least 80%. More preferably, in the reactor, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol product produced by the reactor, before any subsequent processing, such as purification and separation, will typically comprise unreacted acetic acid, ethanol and water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 0 to 20 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

### Integration of Carbonylation and Hydrogenation

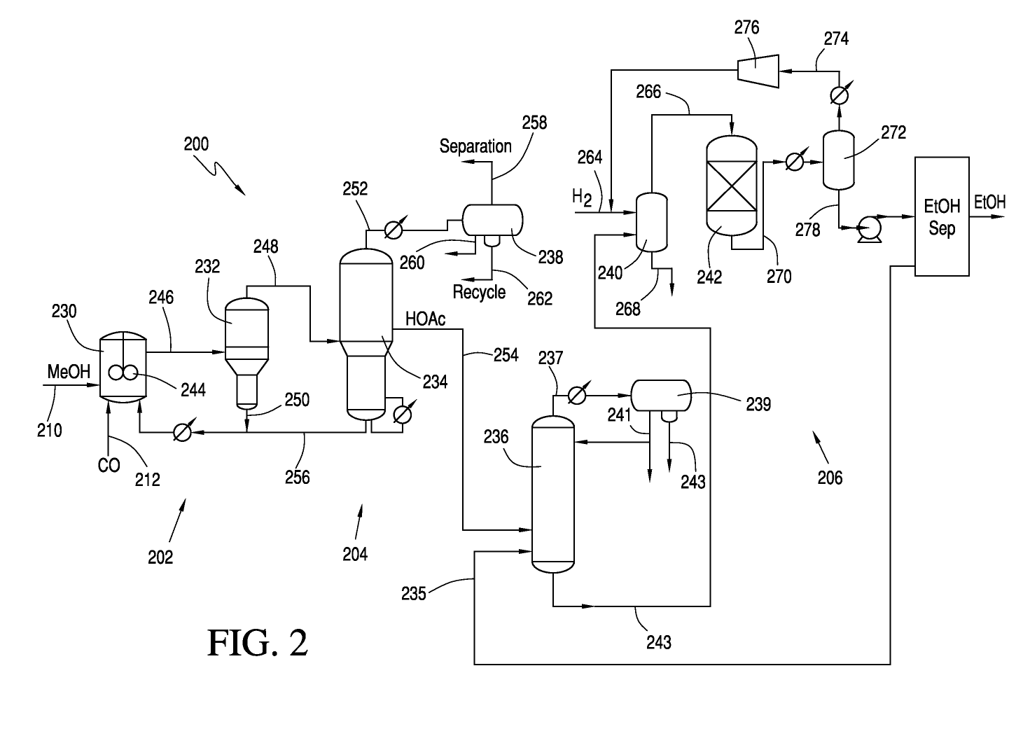

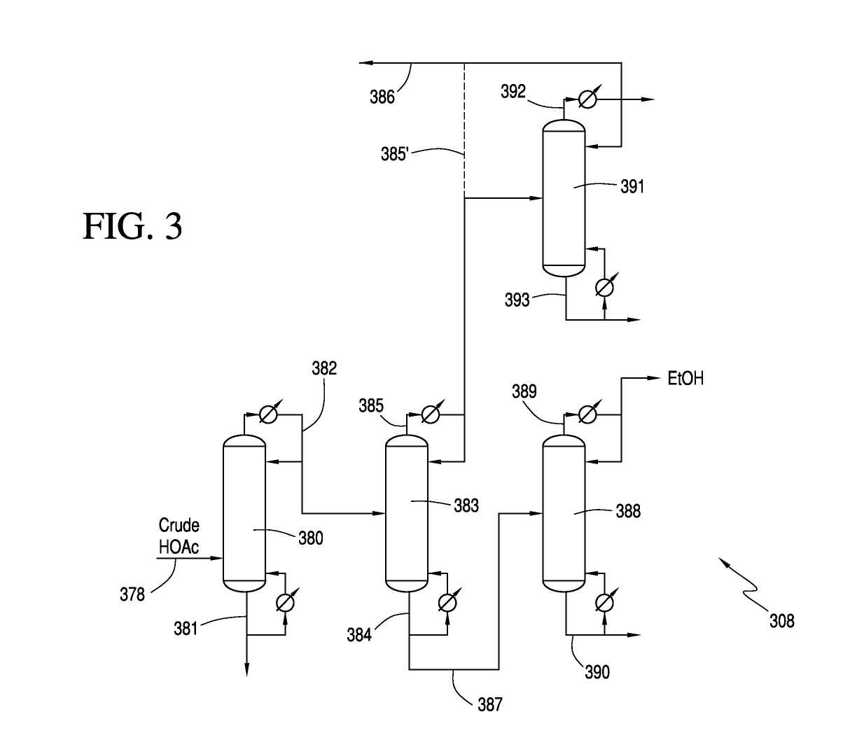

FIG. 2 shows exemplary integrated carbonylation and hydrogenation process 200, which comprises carbonylation reaction zone 202, carbonylation separation zone 204, and hydrogenation reaction zone 206. FIGS. 3-5 show exemplary hydrogenation systems having multiple columns as described herein.

Carbonylation reaction zone 202 comprises carbonylation reactor 230 and flasher 232. Carbonylation separation zone 206 comprises at least one distillation column, e.g., light ends column 234 and/or drying column 236, and phase separator, e.g., decanter, 238. Hydrogenation reaction zone 206 comprises vaporizer 240 and hydrogenation reactor 242.

In carbonylation reaction zone 202, methanol feed stream 210 and carbon monoxide feed stream 212 are fed to a lower portion of carbonylation reactor 230. At least some of the methanol may be converted to, and hence present as, methyl acetate in the liquid reaction composition by reaction with acetic acid product or solvent. The concentration in the liquid reaction composition of methyl acetate is suitably in the range of from 0.5 wt. % to 70 wt. %, e.g., from 0.5 wt. % to 50 wt. %, from 1 wt. % to 35 wt. %, or from 1 wt. % to 20 wt. %.

Carbonylation reactor 230 is preferably either a stirred vessel, e.g., CSTR, or bubble-column type vessel, with agitator 244 or without an agitator, within which the reaction medium is maintained, preferably automatically, at a predetermined level. This predetermined level may remain substantially constant during normal operation. Into reactor 230, methanol, carbon monoxide, and sufficient water may be continuously introduced as needed to maintain at least a finite concentration of water in the reaction medium. In one embodiment, carbon monoxide, e.g., in the gaseous state, is continuously introduced into reactor 230, desirably below agitator 244, which is used to stir the contents. The temperature of reactor 230 may be controlled, as indicated above. Carbon monoxide feed 212 is introduced at a rate sufficient to maintain the desired total reactor pressure.

The gaseous carbon monoxide feed is preferably thoroughly dispersed through the reaction medium by agitator 244. A gaseous purge is desirably vented via an off-gas line (not shown) from reactor 230 to prevent buildup of gaseous by-products, such as methane, carbon dioxide, and hydrogen, and to maintain a carbon monoxide partial pressure at a given total reactor pressure.

The crude acetic acid product is drawn off from reactor 230 at a rate sufficient to maintain a constant level therein and is provided to flasher 232 via stream 246. The crude acetic acid product has the compositions discussed above.

In flasher 232, the crude acetic acid product is separated in a flash separation step to obtain a volatile (“vapor”) overhead stream 248 comprising acetic acid and a less volatile stream 250 comprising a catalyst-containing solution. Impurities from the methanol feed may be passed into overhead stream 248. In one embodiment, overhead stream 248 may be considered a crude acetic acid product, as discussed above. The catalyst-containing solution comprises acetic acid containing rhodium and iodide salt along with lesser quantities of methyl acetate, methyl iodide, and water. The less volatile stream 250 preferably is recycled to reactor 230. Vapor overhead stream 248 also comprises methyl iodide, methyl acetate, water, and permanganate reducing compounds (“PRCs”).

Overhead stream 248 from flasher 232 is directed to carbonylation separation zone 204. Carbonylation separation zone 204 comprises light ends column 234 and decanter 238. Carbonylation separation zone 204 may also comprise additional units, e.g., drying column 236, one or more columns for removing PRCs, heavy ends columns, extractors, etc.

In light ends column 234, stream 248 yields a low-boiling overhead vapor stream 252, a purified acetic acid stream, that preferably is removed via a sidestream 254, and a high boiling residue stream 256. In one embodiment, the acetic acid product that is removed via sidestream 254 preferably is conveyed to drying column 236.

In one embodiment, light ends column 234 may comprise trays having different concentrations of water. In these cases, the composition of a withdrawn sidedraw may vary throughout the column. As such, the withdrawal tray may be selected based on the amount of water that is desired, e.g., more than 0.5 wt %. In another embodiment, the configuration of the column may be varied to achieve a desired amount or concentration of water in a sidedraw. Thus, an acetic acid feed may be produced, e.g., withdrawn from a column, based on a desired water content.

Carbonylation separation zone 204 comprises a second column, such as drying column 236. Sidedraw 254, which is a purified acetic acid stream, may be directed to the second column to separate some of the water from sidedraw 254 as well as other components such as esters and halogens. In addition to sidedraw 254, at least one stream from the hydrogenation process that comprises acetic acid and water in line 235 may also be fed to drying column 236. In one embodiment, at least a portion of the contents of line 235 may be fed to another separation unit in carbonylation zone 204, e.g., light ends columns 234, (not shown in FIG. 2). In one embodiment, at least a portion of the contents of line 235 may be directed to light ends column 234 and/or drying column 236 (not shown in FIG. 2). In one embodiment, sidedraw 254 may be enriched in acetic acid as compared to stream in line 235. Overhead stream 237 from drying column 236 is condensed and biphasically separated in an overhead decanter 239. An aqueous stream in line 241 may be refluxed to drying column 236 and the remaining portion purged as necessary or returned to carbonylation reactor 230. An organic stream in line 243 comprising methyl acetate and/methyl iodide, for example, may be returned to carbonylation reactor 230. In these cases, drying column 236 may yield an acetic acid residue comprising acetic acid and less than 1500 wppm water. Depending on how the drying column is operated, water concentration may be increased to within the range from 0.15 wt. % to 25 wt. %. However, it is preferred to withdraw an acetic acid product in line 245 that contains low amounts of water. In one embodiment, the acetic acid product in line 245 contains less water than stream in line 235. The acetic acid product exiting drying column 236 in line 245 may be fed to hydrogenation reaction zone 206 in accordance with the present invention.

The acetic acid stream, in some embodiments, comprises methyl acetate, e.g., in an amount ranging from 0.01 wt. % to 10 wt. % or from 0.1 wt. % to 5 wt. %. This methyl acetate, in preferred embodiments, may be reduced to form methanol and/or ethanol. In addition to acetic acid, water, and methyl acetate, the purified acetic acid stream may comprise halogens, e.g., methyl iodide, which may be removed from the purified acetic acid stream.

Returning to column 234, low-boiling overhead vapor stream 252 is preferably condensed and directed to an overhead phase separation unit, as shown by overhead receiver decanter 238. Conditions are desirably maintained in the process such that low-boiling overhead vapor stream 252, once in decanter 238, will separate into a light phase and a heavy phase. Generally, low-boiling overhead vapor stream 252 is cooled to a temperature sufficient to condense and separate the condensable methyl iodide, methyl acetate, acetaldehyde and other carbonyl components, and water into two phases. A gaseous portion of stream 252 may include carbon monoxide, and other noncondensable gases such as methyl iodide, carbon dioxide, hydrogen, and the like and is vented from decanter 238 via stream 258.

Condensed light phase 260 from decanter 238 preferably comprises water, acetic acid, and permanganate reducing compounds (“PRCs”), as well as quantities of methyl iodide and methyl acetate. Condensed heavy phase 262 from decanter 238 will generally comprise methyl iodide, methyl acetate, and PRCs. Condensed heavy phase 262, in some embodiments, may be recirculated, either directly or indirectly, to reactor 230. For example, a portion of condensed heavy phase 262 can be recycled to reactor 230, with a slip stream (not shown), generally a small amount, e.g., from 5 to 40 vol. %, or from 5 to 20 vol. %, of condensed heavy phase 262 being directed to a PRC removal system. This slip stream of condensed heavy phase 262 may be treated individually or may be combined with condensed light phase 260 for further distillation and extraction of carbonyl impurities in accordance with one embodiment of the present invention.

Acetic acid sidedraw 254 from column 234 is preferably directed to drying column 236.

In hydrogenation reaction zone 206, hydrogen feed line 264 and acetic acid residue in line 245 are fed to vaporizer 240. Vapor feed stream 266 is withdrawn and fed to hydrogenation reactor 242. In one embodiment, lines 264 and 245 may be combined and jointly fed to the vaporizer 240. The temperature of vapor feed stream 266 is preferably from 100° C. to 350° C., e.g., from 120° C. to 310° C. or from 150° C. to 300° C. Vapor feed stream 266 comprises from 0.15 wt. % to 25 wt. % water. Any feed that is not vaporized is removed from vaporizer 240 via stream 268, as shown in FIG. 2, and may be recycled thereto or discarded. In addition, although FIG. 2 shows line 266 being directed to the top of reactor 242, line 266 may be directed to the side, upper portion, or bottom of reactor 242. Further modifications and additional components to hydrogenation reaction zone 206 are described below.

Reactor 242 contains the catalyst that is used in the hydrogenation of the carboxylic acid, preferably acetic acid. During the hydrogenation process, a crude ethanol product is withdrawn, preferably continuously, from reactor 242 via line 270 and directed to hydrogenation separation zone 208.

Hydrogenation reaction zone 206 comprises flasher 272. Further columns may be included as need to further separate and purify the crude ethanol product as shown in FIGS. 3-5. The crude ethanol product may be condensed and fed to flasher 272, which, in turn, provides a vapor stream and a liquid stream. Flasher 272 may operate at a temperature of from 20° C. to 250° C., e.g., from 30° C. to 250° C. or from 60° C. to 200° C. The pressure of flasher 272 may be from 50 kPa to 2000 kPa, e.g., from 75 kPa to 1500 kPa or from 100 kPa to 1000 kPa. A liquid recycle stream in line 235 from the hydrogenation separation zone may be returned to drying column 236. In some embodiments, line 235 may be combined with line 254 prior to entering drying column 236. In other embodiments, liquid recycle stream in line 235 may be fed directly to drying column 236.

The vapor stream exiting flasher 272 may comprise hydrogen and hydrocarbons, which may be purged and/or returned to hydrogenation reaction zone 206 via line 274. As shown in FIG. 2, the returned portion of the vapor stream passes through compressor 276 and is combined with the hydrogen feed and co-fed to vaporizer 240.

The liquid from flasher 272 is withdrawn and pumped as a feed composition via line 278 to the hydrogenation separation zone 208. Exemplary compositions of line 278 are provided in Table 2. It should be understood that liquid line 278 may contain other components, not listed, such as additional components in the feed.

TABLE 2
FEED COMPOSITION

| | Conc. (wt. %) | Conc. (wt. %) | Conc. (wt. %) |
|---|---|---|---|
| Ethanol | 5 to 72 | 10 to 70 | 15 to 65 |
| Acetic Acid | <90 | 5 to 80 | 0 to 35 |
| Water | 5 to 40 | 5 to 30 | 10 to 26 |
| Ethyl Acetate | <30 | 0.001 to 25 | 1 to 12 |
| Acetaldehyde | <10 | 0.001 to 3 | 0.1 to 3 |
| Acetal | <10 | 0.001 to 6 | 0.01 to 5 |
| Acetone | <5 | 0.0005 to 0.05 | 0.001 to 0.03 |
| Other Alcohols | <5 | <0.005 | <0.001 |
| Other Esters | <5 | <0.005 | <0.001 |
| Other Ethers | <5 | <0.005 | <0.001 |

The amounts indicated as less than (<) in the tables throughout the present application are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 3 may include, but are not limited to, methanol, isopropanol, n-propanol, n-butanol, 2-butanol or mixtures thereof. In one embodiment, the feed composition, e.g., line 262, may comprise propanol, e.g., isopropanol and/or n-propanol, in an amount from 0.001 to 0.1 wt. %, from 0.001 to 0.05 wt. % or from 0.001 to 0.03 wt. %. It should be understood that these other components may be carried through in any of the distillate or residue streams described herein.

Optionally, the crude ethanol product may pass through one or more membranes to separate hydrogen and/or other non-condensable gases. In other optional embodiments, the crude ethanol product may be fed directly to the acid separation column as a vapor feed and the non-condensable gases may be recovered from the overhead of the column.

### Ethanol Separation

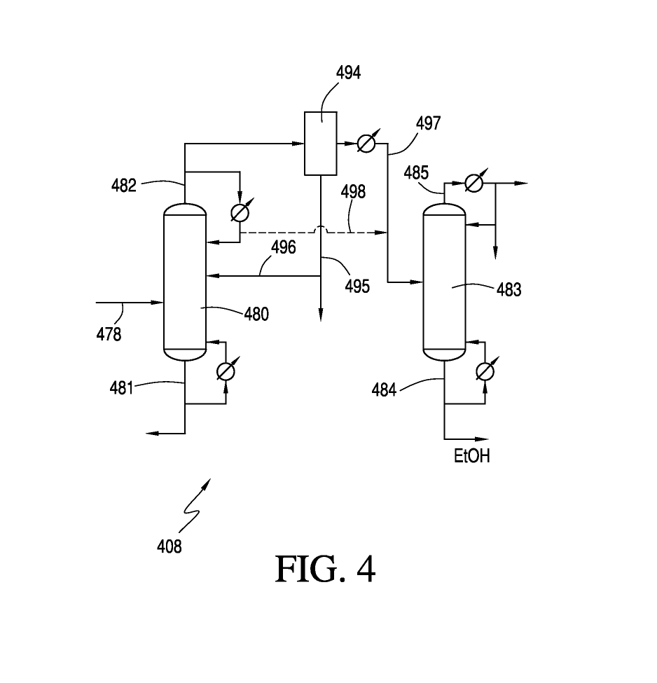

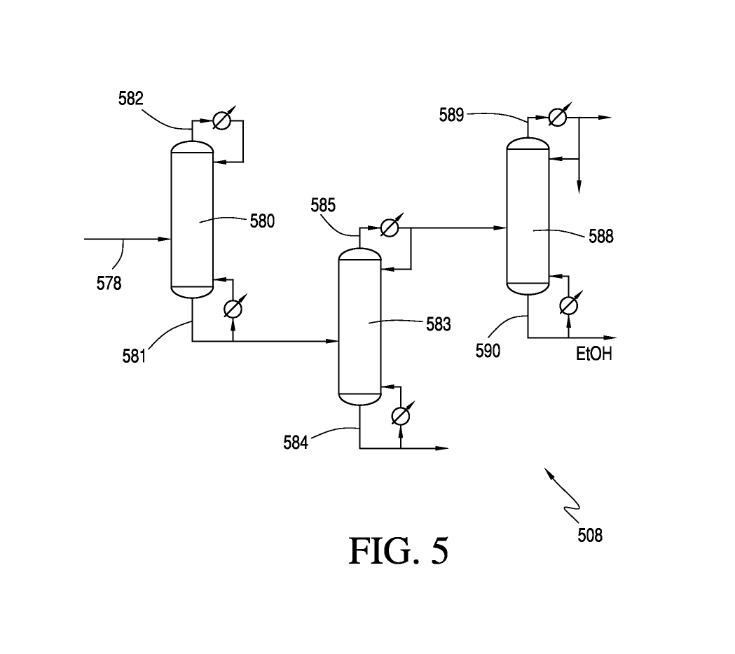

Ethanol produced by the reactor may be recovered using several different techniques. In FIG. 3, the separation of the crude ethanol product uses four columns. In FIG. 4, the crude ethanol product is separated in two columns with an intervening water separation. In FIG. 5, the separation of the crude ethanol product uses three columns. Other separation systems may also be used with embodiments of the present invention.

In one embodiment, feed acetic acid and a liquid recycle stream from hydrogenation separation zone 208 may be mixed prior to vaporizer 240 to form a mixed feed. As stated herein, liquid recycle stream may comprise ethyl acetate. Preferably, the liquid recycle stream is a distillate stream from hydrogenation separation zone 208. Depending on the water concentration of the acetic acid and liquid recycle stream, an optional water stream may be fed directly to vaporizer 240 or may be combined with mixed feed. Hydrogen and the mixed feed may be fed to vaporizer 240 to create a vapor feed stream in line 266 that is directed to reactor 242. Hydrogen feed line 264 may be preheated to a temperature from 30° C. to 150° C., e.g., from 50° C. to 125° C. or from 60° C. to 115° C. Hydrogen feed line 264 may be fed at a pressure from 1300 kPa to 3100 kPa, e.g., from 1500 kPa to 2800 kPa, or 1700 kPa to 2600 kPa.

Vaporizer 240 may operate at a temperature of from 20° C. to 250° C. and at a pressure from 10 kPa to 3000 kPa. Vaporizer 240 produces vapor feed stream in line 266 by transferring the acetic acid, ethyl acetate, and water from the liquid to gas phase below the boiling point of acetic acid in reactor 242 at the operating pressure of the reactor. In one embodiment, the acetic acid in the liquid state is maintained at a temperature below 160° C., e.g., below 150° C. or below 130° C. Vaporizer 240 may be operated at a temperature of at least 118° C.

The temperature of feed stream in line 266 is preferably from 100° C. to 350° C., e.g., from 120° C. to 310° C. or from 150° C. to 300° C. A preheater may be used to further heat feed stream 266 to the reactor temperature.

Any feed that is not vaporized is removed from vaporizer 240 in a blowdown stream and may be recycled or discarded thereto. The mass ratio of feed stream in line 266 to blowdown stream may be from 6:1 to 500:1, e.g., from 10:1 to 500:1, from 20:1 to 500:1 or from 50:1 to 500:1.

In one embodiment, one or more guard beds (not shown) may be used upstream of the reactor, optionally upstream of the vaporizer 240, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

FIG. 3 shows an exemplary hydrogenation separation zone.

In FIG. 3, crude ethanol stream 378 is withdrawn and pumped to the side of first column 380, also referred to as an “acid separation column.” In one embodiment, the contents of ethanol-containing stream 378 are substantially similar to the crude ethanol product obtained from the hydrogenation reactor, except that the composition has been depleted of hydrogen, carbon dioxide, methane and/or ethane, which are removed by the flasher. Accordingly, liquid stream 378 may also be referred to as a crude ethanol product. Exemplary components of liquid stream 378 is similar to Table 2 above.

In the embodiment shown in FIG. 3, line 378 is introduced in the lower part of first column 380, e.g., lower half or lower third. In first column 380, unreacted acetic acid, a portion of the water, and other heavy components, if present, are removed from the composition in line 378 and are withdrawn, preferably continuously, as residue via line 381. Some or all of residue 381 may be returned and/or recycled back to the carbonylation separation zone, e.g., to the drying column 236 and/or the light ends column 234 of the carbonylation zone, e.g., via line 235, as discussed above. Although some of residue in line 381, e.g., a small amount, may be also recycled to vaporizer of hydrogenation reaction zone 206, it preferred that substantially none of the residue in line 381 is directly returned to hydrogenation reaction zone 206. Optionally, at least a portion of residue in line 381 may be purged from the system.

First column 380 also forms an overhead distillate, which is withdrawn in line 382, and which may be condensed and refluxed, for example, at a ratio of from 10:1 to 1:10, e.g., from 3:1 to 1:3 or from 1:2 to 2:1.

When column 380 is operated under standard atmospheric pressure, the temperature of the residue exiting in line 381 preferably is from 95° C. to 120° C., e.g., from 110° C. to 117° C. or from 111° C. to 115° C. The temperature of the distillate exiting in line 382 preferably is from 70° C. to 110° C., e.g., from 75° C. to 95° C. or from 80° C. to 90° C. Column 380 preferably operates at ambient pressure. In other embodiments, the pressure of first column 380 may range from 0.1 kPa to 510 kPa, e.g., from 1 kPa to 475 kPa or from 1 kPa to 375 kPa.

Exemplary components of the distillate and residue compositions for first column 380 are provided in Table 3 below. It should also be understood that the distillate and residue may also contain other components, not listed, such as components in the feed. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 3
ACID COLUMN 380 (FIG. 3)

| | Conc. (wt. %) | Conc. (wt. %) | Conc. (wt. %) |
|---|---|---|---|
| Distillate | | | |
| Ethanol | 20 to 75 | 30 to 70 | 40 to 65 |
| Water | 10 to 40 | 15 to 35 | 20 to 35 |
| Acetic Acid | <2 | 0.001 to 0.5 | 0.01 to 0.2 |
| Ethyl Acetate | <60 | 5.0 to 40 | 10 to 30 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | 60 to 99 | 70 to 95 | 85 to 92 |
| Water | <30 | 1 to 20 | 1 to 15 |
| Ethanol | <1 | <0.9 | <0.07 |

As shown in Table 4, without being bound by theory, it has surprisingly and unexpectedly been discovered that when any amount of acetal is detected in the feed that is introduced to acid separation column 380, the acetal appears to decompose in the column such that less or even no detectable amounts are present in the distillate and/or residue.

The distillate in line 382 preferably comprises ethanol, ethyl acetate, and water, along with other impurities, which may be difficult to separate due to the formation of binary and tertiary azeotropes. To further separate distillate, line 382 is introduced to the second column 383, also referred to as the “light ends column,” preferably in the middle part of column 383, e.g., middle half or middle third. Preferably second column 383 is an extractive distillation column, and an extraction agent is added thereto.

Extractive distillation is a method of separating close boiling components, such as azeotropes, by distilling the feed in the presence of an extraction agent. The extraction agent preferably has a boiling point that is higher than the compounds being separated in the feed. In preferred embodiments, the extraction agent is comprised primarily of water. As indicated above, the first distillate in line 382 that is fed to second column 383 comprises ethyl acetate, ethanol, and water. These compounds tend to form binary and ternary azeotropes, which decrease separation efficiency.

The molar ratio of the water in the extraction agent to the ethanol in the feed to the second column is preferably at least 0.5:1, e.g., at least 1:1 or at least 3:1. In terms of ranges, preferred molar ratios may range from 0.5:1 to 8:1, e.g., from 1:1 to 7:1 or from 2:1 to 6.5:1. Higher molar ratios may be used but with diminishing returns in terms of the additional ethyl acetate in the second distillate and decreased ethanol concentrations in the second column distillate.

In one embodiment, an additional extraction agent, such as water from an external source, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane and chlorinated paraffins, may be added to second column 383. Some suitable extraction agents include those described in U.S. Pat. Nos. 4,379,028, 4,569,726, 5,993,610 and 6,375,807, the entire contents and disclosure of which are hereby incorporated by reference. The additional extraction agent may be combined with a recycled third residue and co-fed to the second column 383. The additional extraction agent may also be added separately to the second column 383. In one aspect, the extraction agent comprises an extraction agent, e.g., water, derived from an external source and none of the extraction agent is derived from the third residue.

Second column 383 may be a tray or packed column. In one embodiment, second column 383 is a tray column having from 5 to 70 trays, e.g., from 15 to 50 trays or from 20 to 45 trays. Although the temperature and pressure of second column 383 may vary, when at atmospheric pressure the temperature of the second residue exiting in line 384 preferably is from 60° C. to 90° C., e.g., from 70° C. to 90° C. or from 80° C. to 90° C. The temperature of the second distillate exiting in line 385 from second column 383 preferably is from 50° C. to 90° C., e.g., from 60° C. to 80° C. or from 60° C. to 70° C. Column 383 may operate at atmospheric pressure. In other embodiments, the pressure of second column 383 may range from 0.1 kPa to 510 kPa, e.g., from 1 kPa to 475 kPa or from 1 kPa to 375 kPa. Exemplary components for the distillate and residue compositions for second column 383 are provided in Table 4 below. It should be understood that the distillate and residue may also contain other components, not listed, such as components in the feed.

TABLE 4
SECOND COLUMN 383 (FIG. 3)

| | Conc. (wt. %) | Conc. (wt. %) | Conc. (wt. %) |
|---|---|---|---|
| Distillate | | | |
| Ethyl Acetate | 10 to 99 | 25 to 95 | 50 to 93 |
| Acetaldehyde | <25 | 0.5 to 15 | 1 to 8 |
| Water | <25 | 0.5 to 20 | 4 to 16 |
| Ethanol | <30 | 0.001 to 15 | 0.01 to 5 |
| Acetal | <5 | 0.001 to 2 | 0.01 to 1 |
| Residue | | | |
| Water | 30 to 90 | 40 to 85 | 50 to 85 |
| Ethanol | 10 to 75 | 15 to 60 | 20 to 50 |
| Ethyl Acetate | <3 | 0.001 to 2 | 0.001 to 0.5 |
| Acetic Acid | <0.5 | 0.001 to 0.3 | 0.001 to 0.2 |

In preferred embodiments, the recycling of the third residue promotes the separation of ethyl acetate from the residue of the second column 383. For example, the weight ratio of ethyl acetate in the second residue to second distillate preferably is less than 0.4:1, e.g., less than 0.2:1 or less than 0.1:1. In embodiments that use an extractive distillation column with water as an extraction agent as the second column 383, the weight ratio of ethyl acetate in the second residue to ethyl acetate in the second distillate approaches zero. Second residue 384 may comprise, for example, from 30% to 99.5% of the water and from 85 to 100% of the acetic acid from line 382. The second distillate in line 385 comprises ethyl acetate and additionally comprises water, ethanol, and/or acetaldehyde. Second distillate 385 may be substantially free of acetic acid. In an optional embodiment, a portion of the second distillate in line 385′ may be combined with line 386, which may be fed, e.g., recycled, to the vaporizer.

The weight ratio of ethanol in the second residue to second distillate preferably is at least 3:1, e.g., at least 6:1, at least 8:1, at least 10:1 or at least 15:1. All or a portion of the third residue is recycled to the second column. In one embodiment, all of the third residue may be recycled until the hydrogenation separation process reaches a steady state and then a portion of the third residue is recycled with the remaining portion being purged from the system. The composition of the second residue will tend to have lower amounts of ethanol than when the third residue is not recycled. As the third residue is recycled, the composition of the second residue, as provided in Table 4, comprises less than 30 wt. % of ethanol, e.g., less than 20 wt. % or less than 15 wt. %. The majority of the second residue preferably comprises water. Notwithstanding this effect, the extractive distillation step advantageously also reduces the amount of ethyl acetate that is sent to the third column, which is highly beneficial in ultimately forming a highly pure ethanol product.

As shown, the second residue from second column 383, which comprises ethanol and water, is fed via line 384 to third column 388, also referred to as the “product column.” More preferably, the second residue in line 384 is introduced in the lower part of third column 388, e.g., lower half or lower third. Third column 388 recovers ethanol, which preferably is substantially pure with respect to organic impurities and other than the azeotropic water content, as the distillate in line 389. The distillate of third column 388 preferably is refluxed as shown in FIG. 3, for example, at a reflux ratio of from 1:10 to 10:1, e.g., from 1:3 to 3:1 or from 1:2 to 2:1. In one embodiment (not shown), a first portion of the third residue in line 390 is recycled to the second column and a second portion is purged and removed from the system. In one embodiment, once the process reaches steady state, the second portion of water to be purged is substantially similar to the amount water formed in the hydrogenation of acetic acid. In one embodiment, a portion of the third residue may be used to hydrolyze any other stream, such as one or more streams comprising ethyl acetate.

Third column 388 is preferably a tray column as described above and operates at atmospheric pressure or optionally at pressures above or below atmospheric pressure. The temperature of the third distillate exiting in line 389 preferably is from 60° C. to 110° C., e.g., from 70° C. to 100° C. or from 75° C. to 95° C. The temperature of the third residue in line 390 preferably is from 70° C. to 115° C., e.g., from 80° C. to 110° C. or from 85° C. to 105° C. Exemplary components of the distillate and residue compositions for third column 388 are provided in Table 5 below. It should be understood that the distillate and residue may also contain other components, not listed, such as components in the feed.

TABLE 5
THIRD COLUMN 388 (FIG. 3)

| | Conc. (wt. %) | Conc. (wt. %) | Conc. (wt. %) |
|---|---|---|---|
| Distillate | | | |
| Ethanol | 75 to 96 | 80 to 96 | 85 to 96 |
| Water | <12 | 1 to 9 | 3 to 8 |
| Acetic Acid | <12 | 0.0001 to 0.1 | 0.005 to 0.05 |
| Ethyl Acetate | <12 | 0.0001 to 0.05 | 0.005 to 0.025 |
| Acetaldehyde | <12 | 0.0001 to 0.1 | 0.005 to 0.05 |
| Diethyl Acetal | <12 | 0.0001 to 0.05 | 0.005 to 0.025 |
| Residue | | | |
| Water | 75 to 100 | 80 to 100 | 90 to 100 |
| Ethanol | <0.8 | 0.001 to 0.5 | 0.005 to 0.05 |
| Ethyl Acetate | <1 | 0.001 to 0.5 | 0.005 to 0.2 |
| Acetic Acid | <2 | 0.001 to 0.5 | 0.005 to 0.2 |

In one embodiment, the third residue in line 390 is withdrawn from third column 388 at a temperature higher than the operating temperature of the second column 383.

Any of the compounds that are carried through the distillation process from the feed or crude reaction product generally remain in the third distillate in amounts of less 0.1 wt. %, based on the total weight of the third distillate composition, e.g., less than 0.05 wt. % or less than 0.02 wt. %. In one embodiment, one or more side streams may remove impurities from any of the columns in the process. Preferably at least one side stream is used to remove impurities from the third column 388. The impurities may be purged and/or retained within the process.

The third distillate in line 389 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit.

Returning to second column 383, the second distillate preferably is refluxed as shown in FIG. 3, optionally at a reflux ratio of 1:10 to 10:1, e.g., from 1:5 to 5:1 or from 1:3 to 3:1. The second distillate in line 385 may be purged or recycled to the reaction zone. In one embodiment, the second distillate in line 385 is further processed in fourth column 391, also referred to as the “acetaldehyde removal column.” In fourth column 391 the second distillate is separated into a fourth distillate, which comprises acetaldehyde, in line 392 and a fourth residue, which comprises ethyl acetate, in line 393. The fourth distillate preferably is refluxed at a reflux ratio of from 1:20 to 20:1, e.g., from 1:15 to 15:1 or from 1:10 to 10:1, and a portion of the fourth distillate is returned to the reaction zone. For example, the fourth distillate may be combined with the acetic acid feed, added to the vaporizer, or added directly to the hydrogenation reactor. The fourth distillate preferably is co-fed with the acetic acid in the feed line to the vaporizer.

Without being bound by theory, since acetaldehyde may be hydrogenated to form ethanol, the recycling of a stream that contains acetaldehyde to the reaction zone increases the yield of ethanol and decreases byproduct and waste generation. In another embodiment, the acetaldehyde may be collected and utilized, with or without further purification, to make useful products including but not limited to n-butanol, 1,3-butanediol, and/or crotonaldehyde and derivatives.

The fourth residue of fourth column 391 may be purged. The fourth residue primarily comprises ethyl acetate and ethanol, which may be suitable for use as a solvent mixture or in the production of esters. In one preferred embodiment, the acetaldehyde is removed from the second distillate in fourth column 391 such that no detectable amount of acetaldehyde is present in the residue of column 391.

Fourth column 391 is preferably a tray column as described above and preferably operates above atmospheric pressure. In one embodiment, the pressure is from 120 kPa to 5,000 kPa, e.g., from 200 kPa to 4,500 kPa, or from 400 kPa to 3,000 kPa. In a preferred embodiment the fourth column 391 may operate at a pressure that is higher than the pressure of the other columns.

The temperature of the fourth distillate exiting in line 392 preferably is from 60° C. to 110° C., e.g., from 70° C. to 100° C. or from 75° C. to 95° C. The temperature of the residue in line 393 preferably is from 70° C. to 115° C., e.g., from 80° C. to 110° C. or from 85° C. to 110° C. Exemplary components of the distillate and residue compositions for fourth column 391 are provided in Table 6 below. It should be understood that the distillate and residue may also contain other components, not listed, such as components in the feed.

TABLE 6
FOURTH COLUMN 391 (FIG. 3)

| | Conc. (wt. %) | Conc. (wt. %) | Conc. (wt. %) |
|---|---|---|---|
| Distillate | | | |
| Acetaldehyde | 2 to 80 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Residue | | | |
| Ethyl Acetate | 40 to 100 | 50 to 100 | 60 to 100 |
| Ethanol | <40 | 0.001 to 30 | 0.01 to 15 |
| Water | <25 | 0.001 to 20 | 2 to 15 |
| Acetaldehyde | <1 | 0.001 to 0.5 | Not detectable |
| Acetal | <3 | 0.001 to 2 | 0.01 to 1 |

In one embodiment, a portion of the third residue in line 390 is recycled to second column 383. In one embodiment, recycling the third residue further reduces the aldehyde components in the second residue and concentrates these aldehyde components in second distillate in line 385 and thereby sent to the fourth column 391, wherein the aldehydes may be more easily separated. The third distillate in line 389 may have lower concentrations of aldehydes and esters due to the recycling of third residue in line 390.

FIG. 4 illustrates another exemplary separation system. In FIG. 4, crude ethanol stream 478 is withdrawn from a hydrogenation reactor and pumped to the side of first column 480. In one preferred embodiment, the hydrogenation reaction zone operates at above 80% acetic acid conversion, e.g., above 90% conversion or above 99% conversion. Thus, the acetic acid concentration in the liquid stream 478 may be low.

Liquid stream 478 is introduced in the middle or lower portion of first column 480, also referred to as acid-water column. For purposes of convenience, the columns in each exemplary separation process, may be referred as the first, second, third, etc., columns, but it is understood that first column 380 in FIG. 3 operates differently than the first column 480 of FIG. 4. In one embodiment, no entrainers are added to first column 480. In FIG. 4, first column 480, water and unreacted acetic acid, along with any other heavy components, if present, are removed from liquid stream 478 and are withdrawn, preferably continuously, as a first residue in line 481. Preferably, a substantial portion of the water in the crude ethanol product that is fed to first column 480 may be removed in the first residue, for example, up to about 75% or to about 90% of the water from the crude ethanol product. Some or all of residue in line 481 may be returned and/or recycled back to the carbonylation separation zone, e.g., to drying column 236 and/or to light ends column 234 of the carbonylation zone via line 235, as discussed above. Although some of residue in line 481, e.g., a small amount, may be also recycled to vaporizer of hydrogenation reaction zone 206, it preferred that substantially none of the residue in line 481 is directly returned to hydrogenation reaction zone 206. Optionally, some of line 481, e.g., a small amount, may be also recycled to vaporizer the hydrogenation reaction zone. Optionally, at least a portion of residue in line 481 may be purged from the system. Reducing the amount of heavies to be purged may improve efficiencies of the process while reducing byproducts. First column 480 also forms a first distillate, which is withdrawn in line 482.

When column 480 is operated under about 170 kPa, the temperature of the residue exiting in line 481 preferably is from 90° C. to 130° C., e.g., from 95° C. to 120° C. or from 100° C. to 115° C. The temperature of the distillate exiting in line 482 preferably is from 60° C. to 90° C., e.g., from 65° C. to 85° C. or from 70° C. to 80° C. In some embodiments, the pressure of first column 480 may range from 0.1 kPa to 510 kPa, e.g., from 1 kPa to 475 kPa or from 1 kPa to 375 kPa.

The first distillate in line 482 comprises water, in addition to ethanol and other organics. In terms of ranges, the concentration of water in the first distillate in line 482 preferably is from less than 20 wt. %, e.g., from 1 wt. % to 19 wt. % or from 5 wt. % to 15 wt. %. A portion of first distillate in line 482 may be condensed and refluxed, for example, at a ratio of from 10:1 to 1:10, e.g., from 3:1 to 1:3 or from 1:2 to 2:1. It is understood that reflux ratios may vary with the number of stages, feed locations, column efficiency and/or feed composition. Operating with a reflux ratio of greater than 3:1 may be less preferred because more energy may be required to operate the first column 480. The condensed portion of the first distillate in line 498 may optionally also be combined with line 497, discussed below, and fed to second column 483.

The remaining portion of the first distillate in 482 is fed to water separation unit 494. Water separation unit 494 may be an adsorption unit, membrane, molecular sieves, extractive column distillation, or a combination thereof. A membrane or an array of membranes may also be employed to separate water from the distillate. The membrane or array of membranes may be selected from any suitable membrane that is capable of removing a permeate water stream from a stream that also comprises ethanol and ethyl acetate.

In a preferred embodiment, water separator 494 is a pressure swing adsorption (PSA) unit. The PSA unit is optionally operated at a temperature from 30° C. to 160° C., e.g., from 80° C. to 140° C., and a pressure of from 0.01 kPa to 550 kPa, e.g., from 1 kPa to 150 kPa. The PSA unit may comprise two to five beds. Water separator 494 may remove at least 95% of the water from the portion of first distillate in line 482, and more preferably from 99% to 99.99% of the water from the first distillate, in a water stream 495. All or a portion of water stream 495 may be returned to column 480 in line 496, where the water preferably is ultimately recovered from column 480 in the first residue in line 481. Additionally or alternatively, all or a portion of water stream 495 may be purged. The remaining portion of first distillate 482 exits the water separator 494 as ethanol mixture stream 497. Ethanol mixture stream 497 may have a low concentration of water of less than 10 wt. %, e.g., less than 6 wt. % or less than 2 wt. %. Exemplary components of ethanol mixture stream 497 and first residue in line 481 are provided in Table 7 below. It should also be understood that these streams may also contain other components, not listed, such as components derived from the feed.

TABLE 7
FIRST COLUMN 480 WITH PSA (FIG. 4)

| | Conc. (wt. %) | Conc. (wt. %) | Conc. (wt. %) |
|---|---|---|---|
| Ethanol Mixture | | | |
| Stream | | | |
| Ethanol | 20 to 95 | 30 to 95 | 40 to 95 |
| Water | <10 | 0.01 to 6 | 0.1 to 2 |
| Acetic Acid | <2 | 0.001 to 0.5 | 0.01 to 0.2 |
| Ethyl Acetate | <60 | 1 to 55 | 5 to 55 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | <90 | 1 to 50 | 2 to 35 |
| Water | 30 to 99 | 45 to 95 | 60 to 90 |
| Ethanol | <1 | <0.9 | <0.3 |

Preferably, ethanol mixture stream 497 is not returned or refluxed to first column 480. The condensed portion of the first distillate in line 498 may be combined with ethanol mixture stream 497 to control the water concentration fed to the second column 483. For example, in some embodiments the first distillate may be split into equal portions, while in other embodiments, all of the first distillate may be condensed or all of the first distillate may be processed in the water separation unit. In FIG. 4, the condensed portion in line 498 and ethanol mixture stream 497 are co-fed to second column 483. In other embodiments, the condensed portion in line 498 and ethanol mixture stream 497 may be separately fed to second column 483. The combined distillate and ethanol mixture has a total water concentration of greater than 0.5 wt. %, e.g., greater than 2 wt. % or greater than 5 wt. %. In terms of ranges, the total water concentration of the combined distillate and ethanol mixture may be from 0.5 to 15 wt. %, e.g., from 2 to 12 wt. %, or from 5 to 10 wt. %.

The second column 483 in FIG. 4, also referred to as the “light ends column,” removes ethyl acetate and acetaldehyde from the first distillate in line 498 and/or ethanol mixture stream 497. Ethyl acetate and acetaldehyde are removed as a second distillate in line 485 and ethanol is removed as the second residue in line 484. Second column 483 may be a tray column or packed column. In one embodiment, second column 483 is a tray column having from 5 to 70 trays, e.g., from 15 to 50 trays or from 20 to 45 trays.

Second column 483 operates at a pressure ranging from 0.1 kPa to 510 kPa, e.g., from 10 kPa to 450 kPa or from 50 kPa to 350 kPa. Although the temperature of second column 483 may vary, when at about 20 kPa to 70 kPa, the temperature of the second residue exiting in line 484 preferably is from 30° C. to 75° C., e.g., from 35° C. to 70° C. or from 40° C. to 65° C. The temperature of the second distillate exiting in line 485 preferably is from 20° C. to 55° C., e.g., from 25° C. to 50° C. or from 30° C. to 45° C.

The total concentration of water fed to second column 483 preferably is less than 10 wt. %, as discussed above. When first distillate in line 498 and/or ethanol mixture stream comprises minor amounts of water, e.g., less than 1 wt. % or less than 0.5 wt. %, additional water may be fed to the second column 483 as an extractive agent in the upper portion of the column. A sufficient amount of water is preferably added via the extractive agent such that the total concentration of water fed to second column 483 is from 1 to 10 wt. % water, e.g., from 2 to 6 wt. %, based on the total weight of all components fed to second column 483. If the extractive agent comprises water, the water may be obtained from an external source or from an internal return/recycle line from one or more of the other columns or water separators.

Suitable extractive agents may also include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof. When extractive agents are used, a suitable recovery system, such as a further distillation column, may be used to recycle the extractive agent.

Exemplary components for the second distillate and second residue compositions for the second column 483 are provided in Table 8, below. It should be understood that the distillate and residue may also contain other components, not listed in Table 8.

TABLE 8
SECOND COLUMN 483 (FIG. 4)

| | Conc. (wt. %) | Conc. (wt. %) | Conc. (wt. %) |
|---|---|---|---|
| Second Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| Second Residue | | | |
| Ethanol | 80 to 99.5 | 85 to 97 | 60 to 95 |
| Water | <20 | 0.001 to 15 | 0.01 to 10 |
| Ethyl Acetate | <1 | 0.001 to 2 | 0.001 to 0.5 |
| Acetic Acid | <0.5 | <0.01 | 0.001 to 0.01 |

The second residue in FIG. 4 comprises one or more impurities selected from the group consisting of ethyl acetate, acetic acid, acetaldehyde, and diethyl acetal. The second residue may comprise at least 100 wppm of these impurities, e.g., at least 250 wppm or at least 500 wppm. In some embodiments, the second residue may contain substantially no ethyl acetate or acetaldehyde.

The second distillate in line 485, which comprises ethyl acetate and/or acetaldehyde, preferably is refluxed as shown in FIG. 4, for example, at a reflux ratio of from 1:30 to 30:1, e.g., from 1:10 to 10:1 or from 1:3 to 3:1. In one aspect, not shown, the second distillate 485 or a portion thereof may be returned to the hydrogenation reactor. The ethyl acetate and/or acetaldehyde in the second distillate may be further reacted in the hydrogenation reactor.

In one embodiment, the second distillate in line 485 and/or a refined second distillate, or a portion of either or both streams, may be further separated to produce an acetaldehyde-containing stream and an ethyl acetate-containing stream. This may allow a portion of either the resulting acetaldehyde-containing stream or ethyl acetate-containing stream to be recycled to the hydrogenation reactor while purging the other stream. The purge stream may be valuable as a source of either ethyl acetate and/or acetaldehyde.

FIG. 5 illustrates another exemplary separation system. In FIG. 5, crude ethanol stream 578 is withdrawn from a hydrogenation reactor and pumped to the side of first column 580. In one preferred embodiment, the hydrogenation reaction zone operates at above 80% acetic acid conversion, e.g., above 90% conversion or above 99% conversion. Thus, the acetic acid concentration in the liquid stream 578 may be low.

In the exemplary embodiment shown in FIG. 5, liquid stream 578 is introduced in the lower part of first column 580, e.g., lower half or middle third. In one embodiment, no entrainers are added to first column 580. In first column 580, a weight majority of the ethanol, water, acetic acid, and other heavy components, if present, are removed from liquid stream 578 and are withdrawn, preferably continuously, as residue in line 581.

First column 580 also forms an overhead distillate, which is withdrawn in line 582, and which may be condensed and refluxed, for example, at a ratio of from 30:1 to 1:30, e.g., from 10:1 to 1:10 or from 1:5 to 5:1. The overhead distillate in stream 582 preferably comprises a weight majority of the ethyl acetate from liquid stream 578. Overhead distillate in stream 582 may be combined with a recycle line from column 583 as discussed below, and returned to the hydrogenation reaction zone.

When column 580 is operated under about 170 kPa, the temperature of the residue exiting in line 581 preferably is from 70° C. to 155° C., e.g., from 90° C. to 130° C. or from 100° C. to 110° C. The base of column 580 may be maintained at a relatively low temperature by withdrawing a residue stream comprising ethanol, water, and acetic acid, thereby providing an energy efficiency advantage. The temperature of the distillate exiting in line 582 from column 580 preferably at 170 kPa is from 75° C. to 100° C., e.g., from 75° C. to 83° C. or from 81° C. to 84° C. In some embodiments, the pressure of first column 580 may range from 0.1 kPa to 510 kPa, e.g., from 1 kPa to 475 kPa or from 1 kPa to 375 kPa. Exemplary components of the distillate and residue compositions for first column 580 are provided in Table 10 below. It should also be understood that the distillate and residue may also contain other components, not listed in Table 9.

TABLE 9
FIRST COLUMN 580 (FIG. 5)

| | Conc. (wt. %) | Conc. (wt. %) | Conc. (wt. %) |
|---|---|---|---|
| Distillate | | | |
| Ethyl Acetate | 10 to 85 | 15 to 80 | 20 to 75 |
| Acetaldehyde | 0.1 to 70 | 0.2 to 65 | 0.5 to 65 |
| Acetal | <0.1 | <0.1 | <0.05 |
| Acetone | <0.05 | 0.001 to 0.03 | 0.01 to 0.025 |
| Ethanol | 3 to 55 | 4 to 50 | 5 to 45 |
| Water | 0.1 to 20 | 1 to 15 | 2 to 10 |
| Acetic Acid | <2 | <0.1 | <0.05 |
| Residue | | | |
| Acetic Acid | 0.01 to 35 | 0.1 to 30 | 0.2 to 25 |
| Water | 5 to 40 | 10 to 35 | 15 to 30 |
| Ethanol | 10 to 75 | 15 to 70 | 20 o 65 |

In one embodiment of the present invention, column 580 may be operated at a temperature where most of the water, ethanol, and acetic acid are removed from the residue stream and only a small amount of ethanol and water is collected in the distillate stream due to the formation of binary and tertiary azeotropes. The weight ratio of water in the residue in line 581 to water in the distillate in line 582 may be greater than 1:1, e.g., greater than 2:1. The weight ratio of ethanol in the residue to ethanol in the distillate may be greater than 1:1, e.g., greater than 2:1

The amount of acetic acid in the first residue may vary depending primarily on the conversion in the hydrogenation reactor. In one embodiment, when the conversion is high, e.g., greater than 90%, the amount of acetic acid in the first residue may be less than 10 wt. %, e.g., less than 5 wt. % or less than 2 wt. %. In other embodiments, when the conversion is lower, e.g., less than 90%, the amount of acetic acid in the first residue may be greater than 10 wt. %.

The distillate preferably is substantially free of acetic acid, e.g., comprising less than 1000 wppm, less than 500 wppm or less than 100 wppm acetic acid. The distillate may be purged from the system or recycled in whole or part to the hydrogenation reactor. In some embodiments, the distillate may be further separated, e.g., in a distillation column (not shown), into an acetaldehyde stream and an ethyl acetate stream. Either of these streams may be returned to the hydrogenation reactor or separated as a separate product.

Some species, such as acetals, may decompose in first column 580 such that very low amounts, or even no detectable amounts, of acetals remain in the distillate or residue.

To recover ethanol, the residue in line 581 may be further separated in second column 583, also referred to as an “acid separation column.” An acid separation column may be used when the acetic acid concentration in the first residue is greater than 1 wt. %, e.g., greater than 5 wt. %. The first residue in line 581 is introduced to second column 583 preferably in the top part of column 583, e.g., top half or top third. Second column 583 yields a second residue in line 584 comprising acetic acid and water, and a second distillate in line 585 comprising ethanol. Some or all of residue in line 584 may be returned and/or recycled back to the carbonylation separation zone, e.g., to drying column 235 and/or to light ends column 234 of carbonylation zone 236, as discussed above. Although some of residue in line 584, e.g., a small amount, may be also recycled to vaporizer of hydrogenation reaction zone 206, it preferred that substantially none of the residue in line 584 is directly returned to hydrogenation reaction zone 206. Optionally, at least a portion of residue in line 584 may be purged from the system.

Second column 583 may be a tray column or packed column. In one embodiment, second column 583 is a tray column having from 5 to 150 trays, e.g., from 15 to 50 trays or from 20 to 45 trays. Although the temperature and pressure of second column 583 may vary, when at atmospheric pressure the temperature of the second residue exiting in line 584 preferably is from 95° C. to 130° C., e.g., from 100° C. to 125° C. or from 110° C. to 120° C. The temperature of the second distillate exiting in line 585 preferably is from 60° C. to 105° C., e.g., from 75° C. to 100° C. or from 80° C. to 100° C. The pressure of second column 583 may range from 0.1 kPa to 510 kPa, e.g., from 1 kPa to 475 kPa or from 1 kPa to 375 kPa. Exemplary components for the distillate and residue compositions for second column 583 are provided in Table 10 below. It should be understood that the distillate and residue may also contain other components, not listed in Table 10.

TABLE 10
SECOND COLUMN 583 (FIG. 5)

| | Conc. (wt. %) | Conc. (wt. %) | Conc. (wt. %) |
|---|---|---|---|
| Second Distillate | | | |
| Ethanol | 70 to 99.9 | 75 to 98 | 80 to 95 |
| Ethyl Acetate | <10 | 0.001 to 5 | 0.01 to 3 |
| Acetaldehyde | <5 | 0.001 to 1 | 0.005 to 0.5 |
| Water | 0.1 to 30 | 1 to 25 | 5 to 20 |
| Second Residue | | | |
| Acetic Acid | 0.1 to 45 | 0.2 to 40 | 0.5 to 35 |
| Water | 45 to 99.9 | 55 to 99.8 | 65 to 99.5 |
| Ethyl Acetate | <2 | <1 | <0.5 |
| Ethanol | <5 | 0.001 to 5 | <2 |

The weight ratio of ethanol in the second distillate in line 585 to ethanol in the second residue in line 584 preferably is at least 35:1. In one embodiment, the weight ratio of water in the second residue 584 to water in the second distillate 585 is greater than 2:1, e.g., greater than 4:1 or greater than 6:1. In addition, the weight ratio of acetic acid in the second residue 584 to acetic acid in the second distillate 585 preferably is greater than 10:1, e.g., greater than 15:1 or greater than 20:1. Preferably, the second distillate in line 585 is substantially free of acetic acid and may only contain, if any, trace amounts of acetic acid.

As shown, the second distillate in line 585 is fed to a third column 588, e.g., ethanol product column, for separating the second distillate into a third distillate (ethyl acetate distillate) in line 589 and a third residue (ethanol residue) in line 590. Second distillate in line 585 may be introduced into the lower part of column 588, e.g., lower half or lower third. Third distillate 589 is preferably refluxed, for example, at a reflux ratio greater than 2:1, e.g., greater than 5:1 or greater than 10:1. Additionally, at least a portion of third distillate 589 may be purged. Third column 588 is preferably a tray column as described herein and preferably operates at atmospheric pressure. The temperature of the third residue exiting from third column 588 preferably is from 60° C. to 110° C., e.g., from 70° C. to 100° C. or from 75° C. to 95° C. The temperature of the third distillate exiting from third column 588 preferably is from 70° C. to 115° C., e.g., from 80° C. to 110° C. or from 85° C. to 105° C., when the column is operated at atmospheric pressure.

The remaining water from the second distillate in line 585 may be removed in further embodiments of the present invention. Depending on the water concentration, the ethanol product may be derived from the second distillate in line 585. Some applications, such as industrial ethanol applications, may tolerate water in the ethanol product, while other applications, such as fuel applications, may require an anhydrous ethanol. The amount of water in the distillate of line 585 may be closer to the azeotropic amount of water, e.g., at least 4 wt. %, preferably less than 20 wt. %, e.g., less than 12 wt. % or less than 7.5 wt. %. Water may be removed from the second distillate in line 585 using several different separation techniques as described herein. Particularly preferred techniques include the use of distillation column, membranes, adsorption units, and combinations thereof.

The columns shown in FIGS. 3-5 may comprise any distillation column capable of performing the desired separation and/or purification. Each column preferably comprises a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these zones although in some embodiments subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The ethanol product produced by the process of the present invention may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 11.

TABLE 11
FINISHED ETHANOL COMPOSITIONS

| Component | Conc. (wt. %) | Conc. (wt. %) | Conc. (wt. %) |
|---|---|---|---|
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit as discussed above. In such embodiments, the ethanol concentration of the ethanol product may be higher than indicated in Table 12, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entireties of which is incorporated herein by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Yin U.S. Pat. No. 3,130,007, the entireties of which are hereby incorporated herein by reference.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising the steps of:
(a) hydrogenating acetic acid obtained from a carbonylation system in the presence of a catalyst in a hydrogenation reactor under conditions effective to form a crude ethanol product;
(b) separating, in at least one column, at least a portion of the crude ethanol product into a distillate comprising ethanol, water, and ethyl acetate, and a residue comprising acetic acid and water;
(c) directing at least a portion of the residue to at least one column of the carbonylation system; and
(d) separating the distillate to form a purified ethanol product.

2. The process of claim 1, wherein substantially none of the residue is directly fed to the hydrogenation reactor.

3. The process of claim 1, wherein the at least one column of the carbonylation system comprises a carbonylation system drying column.

4. The process of claim 3, further comprising the step of:
separating, in the drying column, the residue to form a purified acetic acid stream and a water stream.

5. The process of claim 4, wherein the purified acetic acid stream comprises less than 1500 wppm water.

6. The process of claim 1, wherein the residue comprises:
from 60 wt. % to 99 wt. % acetic acid; and
from 1 wt. % to 30 wt. % water.

7. The process of claim 1, wherein the residue comprises:
from 1 wt. % to 70 wt. % acetic acid; and
from 30 wt. % to 99 wt. % water.

8. The process of claim 1, wherein the residue comprises:
from 0.1 wt. % to 45 wt. % acetic acid; and
from 45 wt. % to 99.9 wt. % water.

9. A process for producing ethanol, comprising the steps of:
(a) reacting carbon monoxide with at least one reactant in a first reactor containing a reaction medium to produce a liquid reaction product comprising acetic acid, wherein the reaction medium comprises water, acetic acid, methyl acetate, a halogen promoter, and a first catalyst;
(b) separating the reaction product in a flasher into a liquid recycle stream and a vapor stream comprising acetic acid, the halogen promoter, methyl acetate, water, and mixtures thereof;
(c) separating the vapor stream in a first column to yield a first overhead stream comprising methyl acetate, acetaldehyde, the halogen promoter, water, and mixtures thereof, an acetic acid product sidedraw, and an optional first residue stream;
(d) separating the acetic acid product sidedraw in a second column to yield a second overhead stream comprising water, methyl acetate, the halogen promoter, and mixtures thereof and a second residue comprising acetic acid;
(e) hydrogenating acetic acid from a portion of the second residue in a second reactor in the presence of a catalyst under conditions effective to form a crude ethanol product;
(f) separating at least a portion of the crude ethanol product in a third column to yield a third overhead comprising ethanol, water, and ethyl acetate, and a third residue comprising acetic acid and less than 30 wt. % water;
(g) directing at least a portion of the third residue to the first column and/or second column; and
(h) recovering ethanol from the third overhead.

10. The process of claim 9, wherein the second residue comprises less than 1500 wppm water.

11. The process of claim 9, wherein the second residue comprises:
from 60 wt. % to 99 wt. % acetic acid; and
from 1 wt. % to 30 wt. % water.

12. The process of claim 9, further comprising:
separating the third overhead in a fourth columns to yield a fourth distillate comprising ethyl acetate and aldehyde, and a fourth residue comprising ethanol and water; and
separating the fourth residue in a fifth columns to yield a fifth distillate comprising ethanol and a fifth residue comprising water.

13. A process for producing ethanol, comprising the steps of:
(a) reacting carbon monoxide with at least one reactant in a first reactor containing a reaction medium to produce a liquid reaction product comprising acetic acid, wherein the reaction medium comprises water, acetic acid, methyl acetate, a halogen promoter, and a first catalyst;
(b) separating the reaction product in a flasher into a liquid recycle stream and a vapor stream comprising acetic acid, the halogen promoter, methyl acetate, water, and mixtures thereof;
(c) separating the vapor stream in a first column to yield a first overhead stream comprising methyl acetate, acetaldehyde, the halogen promoter, water and mixtures thereof, an acetic acid product sidedraw, and an optional first residue stream;
(d) separating the acetic acid product sidedraw in a second column to yield a second overhead stream comprising water, methyl acetate, the halogen promoter, and mixtures thereof and a second residue comprising acetic acid;
(e) hydrogenating acetic acid from a portion of the second residue in a second reactor in the presence of a catalyst under conditions effective to form a crude ethanol product;
(f) separating at least a portion of the crude ethanol product in a third column to yield a third residue comprising acetic acid and a substantial portion of the water and a third overhead comprising ethanol, ethyl acetate, and water;
(g) removing water from at least a portion of the third overhead to yield an ethanol mixture stream comprising less than 10 wt. % water; and
(h) separating a portion of the ethanol mixture stream in a fourth column to yield a fourth residue comprising ethanol and a fourth overhead comprising ethyl acetate; and
(i) directing at least a portion of the third residue to the first column and/or the second column.

14. The process of claim 13, wherein the second residue comprises less than 1500 wppm water.

15. The process of claim 13, wherein the second residue comprises:
from 1 wt. % to 70 wt. % acetic acid; and
from 30 wt. % to 99 wt. % water.

16. The process of claim 13, further comprising
removing water from at least a portion of the third distillate to yield an ethanol mixture stream comprising less than 10 wt. % water;
separating a portion of the ethanol mixture stream in a fourth distillation column to yield a fourth residue comprising ethanol and a fourth distillate comprising ethyl acetate

17. A process for producing ethanol, comprising the steps of:
(a) reacting carbon monoxide with at least one reactant in a first reactor containing a reaction medium to produce a liquid reaction product comprising acetic acid, wherein the reaction medium comprises water, acetic acid, methyl acetate, a halogen promoter, and a first catalyst;
(b) separating the reaction product in a flasher into a liquid recycle stream and a vapor stream comprising acetic acid, the halogen promoter, methyl acetate, water, and mixtures thereof;
(c) separating the vapor stream in a first column to yield a first overhead stream comprising methyl acetate, acetaldehyde, the halogen promoter, water, and mixtures thereof, an acetic acid product sidedraw, and an optional first residue stream;
(d) separating the acetic acid product sidedraw in a second column to yield a second overhead stream comprising water, methyl acetate, the halogen promoter, and mixtures thereof and a second residue comprising acetic acid;
(e) hydrogenating acetic acid from a portion of the second residue in a second reactor the presence of a catalyst under conditions effective to form a crude ethanol product;
(f) separating a portion of the crude ethanol product in a third column to yield a third overhead comprising ethyl acetate and acetaldehyde, and a third residue comprising ethanol, ethyl acetate, acetic acid and water;
(g) separating a portion of the first residue in a fourth column to yield a fourth residue comprising acetic acid and water and a fourth overhead comprising ethanol and ethyl acetate; and
(i) directing at least a portion of the fourth residue to the first and/or second column.

18. The process of claim 17, further comprising the steps of separating a portion of the fourth overhead in a fifth column to yield a fifth residue comprising ethanol and a fifth overhead comprising ethyl acetate.

19. The process of claim 17, wherein the second residue comprises less than 1500 wppm water.

20. The process of claim 17, wherein the residue comprises:
from 0.1 wt. % to 45 wt. % acetic acid; and
from 45 wt. % to 99.9 wt. % water.
